(19) United States
(12) Patent Application Publication
(10) Pub. No.: US 2005/0135868 A1
(43) Pub. Date: Jun. 23, 2005

(54) Automotive interior liquid applicator

(76) Inventors: Wen-Chen Su, Lexington, KY (US); Todd Colburn, La Jolla, CA (US); Frederick Large, Carlsbad, CA (US); Dan Anderson, Carlsbad, CA (US); Steven Ruble, Lexington, KY (US); Mark D. Koenig, Lexington, KY (US); Brooke T. Baxter, New Milford, CT (US); William R. Bucknam JR., Woodbury, CT (US)

Correspondence Address: FULWIDER PATTON LEE & UTECHT, LLP, 200 OCEANGATE, SUITE 1550, LONG BEACH, CA, 90802, US

(21) Appl. No.: 10/990,209

(22) Filed: Nov. 16, 2004

Related U.S. Application Data
(63) Continuation-in-part of application No. 10/437,762, filed on May 14, 2003, now Pat. No. 6,817,801.

(51) Int. Cl. B43K 5/00; A61F 13/15; B43M 11/06

(52) U.S. Cl. 401/205; 401/186

(57) ABSTRACT

An applicator device for applying treatment fluid to various interior surfaces such as those found in an automobile, which is constructed with an applicator head including a housing and a flow chamber and an applicator pad affixed to the housing. The applicator head is further configured to complementally and releasably receive an associated fluid container.

### CROSS-REFERENCES TO RELATED APPLICATIONS

This application is a continuation-in-part of application Ser. No. 10/437,762, filed May 14, 2003, which issued as U.S. Pat. No. ______ and on which this application claims priority under 35 U.S.C. § 120.

### BACKGROUND OF THE INVENTION

1. Field of the Invention

The present invention relates to an applicator device for conveniently and effectively applying cleaning and other treatment fluids to a variety of surfaces, such as a dashboard or the many other upholstery surfaces found in the interior of an automobile.

2. Description of Related Art

Automobile and other vehicle owners often use various cleaning, polishing and other appearance maintenance substances to enhance and preserve the internal appearance of their vehicles. These substances may be found in a multiplicity of chemical compositions assuming several forms, and will generally be sprayed or squeezed from their container directly onto the surface to be treated or onto a simple applicator device such as a rag or sponge. Such devices, however, have their disadvantages. For instance, a used rag soaked with treatment liquid must be either discarded or laundered after use. Laundering may prove to be time consuming and expensive, and discarding the rag and purchasing a new one for each use can be inconvenient, cost prohibitive and detrimental to the environment. Also, traditional applicator devices, such as sponges or rags, are not easily or efficiently manipulated by the user, and are often not shaped to conform to and reach the many and varied contours and crevices in an automobile interior.

Cleaning or other treatment fluids often come in contact with the user's hands, causing them to be dirtied or otherwise harmed by such contact. A sponge, when gripped, may also become distorted in the middle to curve upwardly at the sides as the user squeezes it or attempts to apply controlled and focused pressure to a certain spot, resulting in an uneven and distorted contact surface that negates the smooth flow and even application of substance being applied. Furthermore, with traditional sponges or rag applicators, the user must periodically apply cleaning or treatment fluid to the applicator.

Many devices have been developed for applying polishing, waxing, cleaning or other treatment compounds to a surface. However, without a handle or other design measures to assist the user in focusing and controlling the amount and magnitude of his or her treatment or cleaning efforts, traditional applicator devices have proven to be inconvenient and inefficient, especially for treatment of automobile upholstering or dash boards. What adds to the challenge of applying these fluids to the interior surfaces of a automobile is the fact that such surfaces are often formed in recesses or are configured with compound curvatures, angles and crevices of various shapes and sizes that challenge the effective and sustained access and control achievable with conventional applicators. For example, when using many traditional applicators, a user may encounter significant difficulty when attempting to apply treatment fluid to the portion of an automobile dashboard that is directly adjacent to its intersection with the rearwardly sloped windshield. Additionally, without a readily accessible resupply of such cleaning or treatment fluid, even with easily reachable surfaces, continuous re-application of fluid to the treatment surface or applicator device leads to inefficient expenditure of a user's time and energy. Therefore, an applicator device is needed that can provide for a steady, prolonged and efficient flow of treatment fluid that is well distributed across the lateral and longitudinal dimensions of the working surface, but that is also capable of reaching the totality of the surfaces found in an automobile's interior.

Several prior art devices have proposed the basic concepts of a porous applicator fixably mounted to some type of a container having a reservoir or breakable bladder to hold the fluid to be applied therein. The fluid contained within the container of these devices is absorbed into the porous applicator, and the applicator is then applied to a solid surface to distribute the fluid thereon. Because such devices often lack the requisite dispensing capabilities for controlled amounts of fluid over an extended surface area of the applicator pad, they often simply serve to distribute fluid to a central location on the pad, which may result in a concentration of fluid in its center and an insufficient amount at the forward, rear and lateral extremities thereof. Furthermore, the contact surfaces of the applicator pads of such devices are often not adapted to conform to and/or reach the wide array of surfaces found in a conventional automobile, and such devices may be unsuitable or unadaptable for application of different fluids that are designed for use with differing types of respective surface materials, such as leather, vinyl and the like. In addition, the relatively small surface area of some such applicators may make application to an automobile time consuming and laborious.

In recognition of some of the aforementioned shortcomings, a wax applicator has been proposed which includes a flat applicator plate having a central opening therein and a porous pad mounted thereunder and formed with a centrally disposed communication opening. A cylindrical handle forms a liquid wax receiving container and is formed on one end with a coupling plate. The coupling plate is formed with a central opening alignable with the openings in the applicator plate and pad. A domed valve is mounted over such outlet opening to, upon compression of the walls of the handle, release charges of liquid wax to be dispensed directly through the opening in the pad to the underlying surface to be waxed. A device of this type is marketed under the trademark Quick n' Neat™ by Clean Shot Products Co., of Emporia, Kans. Such devices fail to provide for distribution of the dispensed liquid throughout the surface of the applicator pad thus inhibiting efforts to provide for broad, uniform application of treatment fluid, and require a certain degree of dexterity and effort to reach and properly apply treatment fluid to the less accessible interior areas of a typical automobile.

A need exists in the marketplace for an applicator device capable of sustained and controlled application of a desired treatment fluid in a uniform manner to the many and varied surfaces found in the interior of an automobile. It would also be especially beneficial if the housing that mounts the applicator's pad was designed for rapid and secure mating with a complementally designed replaceable container. The present invention fulfils this need.

### SUMMARY OF THE INVENTION

Briefly and in general terms, the present invention is directed to an applicator device for spreading and applying cleaning, protecting or other treatment fluids to a wide array of variously shaped and dimensioned surfaces, such as those found in the interior of an automobile. The applicator device includes a container enclosing a reservoir having a ready supply of treatment fluid that also serves as a handle by which the user grasps the applicator device.

Joined to the container is a complementally mating applicator head comprising an applicator pad and a dispenser housing including a flow chamber. The applicator pad is affixed or otherwise attached to the bottom surface of the housing, to which the applicator pad is attached or otherwise affixed at an attachment surface. In one preferred embodiment, the fluid is transferred through the housing to its bottom surface by the flow chamber for delivery to various desired portions on the attachment surface of the applicator pad. In another embodiment, the housing's bottom surface may be defined by a distribution plate having a distribution surface formed with at least one distribution channel, which may also or alternatively be correspondingly formed on the applicator pad attachment surface, which then facilitates the flow of fluid to various desired portions of the applicator pad. In such an embodiment, the distribution may also be achieved by passages or channels formed in a plate or the like sandwiched into the interface between the distribution plate and the pad. In another permutation, the flow chamber works in conjunction with a plurality of dispensing openings arrayed about the distribution plate to dispense the fluid of the container to the applicator's pad for further transfer therethrough to the pad's working surface. In another permutation incorporating a distribution plate that defines the bottom surface of the housing, the plate may include a central manifold from which distribution channels extend outwardly and forwardly to distribute the fluid across the width and length of the applicator's pad.

For joining the container to the applicator head, various configurations are contemplated, and in one preferred embodiment, the dispenser housing includes a somewhat funnel shaped upwardly and rearwardly opening cowling disposed about an inlet device, with the inlet device further including a coupling shell for releasably receiving the neck of the container by way of a snap lock, bayonet fit, bead and flange, threaded engagement or other appropriate connection. The housing is configured with its inlet device and cowling angling upwardly and rearwardly at a predetermined angle relative to the bottom surface of the housing such that the elongated body of the container projects longitudinally of the inlet device at the same predetermined angle when the container is coupled to the housing. When so configured, the container, inlet, flow chamber and distribution plate, if present, cooperate to form a fluid communication path therethrough to the applicator pad. A flow control, which in one preferred embodiment is in the form of a one way valve, is positioned at some point along this communication path to regulate the flow of fluid to the applicator pad.

The present invention may take the form of several embodiments designed for application of treatment and cleaning fluid to a variety of interior surfaces as may be found in an automobile, and may be adapted for each by, for example, modifying the surface area, shape and material composition of the applicator pad, or the material composition of the fluid in the container. In a preferred embodiment, the applicator pad is generally flat iron shaped, having similar dimensions to those of the housing's bottom surface, and may be formed with a forwardly projecting flexible finger to further facilitate the application of fluid to hard to reach surfaces.

In a related aspect of the invention, the lateral edges of the outer perimeter of the applicator pad may extend laterally outwardly from the bottom surface of the housing, and may be oriented generally transversely to the longitudinal axis of the pad's attachment and working surfaces. However, it is also contemplated that the sides of the applicator pad may angle downwardly and outwardly from the attachment surface to culminate in a working surface having a similar general shape, but a relatively greater surface area than that of the attachment surfaces of the applicator pad and the housing's bottom surface. In yet another preferred embodiment, the side walls of the dispenser housing may be formed on their lower extremities with respective laterally projecting side wings to define a distribution plate of relatively greater surface area than in the above described embodiment.

In one preferred embodiment, the container may be disposable and replaceable, being produced in multiple variants adapted to contain any number of specific use fluids, such as those designed for cleaning or treating vinyl, leather and the like. However, it is also contemplated that the container may be refillable by a filling stem projecting outwardly from its proximal end.

In still another preferred embodiment seeking to emphasize a comfortable interaction with the hand of the user, the container may be formed with at least an ergonomically adapted dorsal wall designed to be complementally received in the user's palm, and may include finger grooves for receipt of the fingers of the user's grasping hand. Also in keeping with the invention, the container may take the form of a squeeze tube or other appropriate structure formed with flexible walls, whereby squeezing of the walls urges the flow of fluid along the fluid communication path, through the flow control, and to the applicator pad. In another possible aspect of the invention, the container may be formed with rigid walls requiring the user to elevate the container above the level of the dispenser housing to initiate fluid flow through the housing.

These and other features and advantages of the applicator device will become apparent from the following detailed description of preferred embodiments which, taken in conjunction with the accompanying drawings, illustrate by way of example the principles of the invention.

### BRIEF DESCRIPTION OF THE DRAWINGS

FIG. 1 is a perspective broken view of an applicator device embodying the present invention;

FIG. 2 is a front view of the applicator device shown in FIG. 1;

FIG. 3 is a top view of the applicator device shown in FIG. 1;

FIG. 4 is a bottom view of the applicator device shown in FIG. 1;

FIG. 5 is a left-hand end view of the applicator device shown in FIG. 1;

FIG. 6 is a right-hand end view of the applicator device shown in FIG. 1;

FIG. 7 is a longitudinal sectional view, in enlarged scale, taken along line 7-7 of FIG. 3;

FIG. 7a is a transverse sectional view, in enlarged scale, taken along line 7A-7A of FIG. 7;

FIG. 8 is a horizontal sectional view taken along line 8-8 of FIG. 7;

FIG. 9 is a vertical sectional view taken along line 9-9 of FIG. 7;

FIG. 10 is a partial horizontal sectional view, in an enlarged scale, of the flow control mechanism shown in FIG. 8;

FIG. 11 is a vertical sectional view taken along line 11-11 of FIG. 11;

FIG. 12 is a transverse sectional view, in an enlarged scale, taken along the line 12-12 of FIG. 7;

FIG. 13 is a transverse sectional view, in an enlarged scale, taken along the line 12-12 of FIG. 7 similar to FIG. 12;

FIG. 14 is a longitudinal sectional view, in an enlarged scale, of a portion of the container coupling assembly included in the device shown in FIG. 7;

FIG. 15 is a longitudinal sectional view similar to FIG. 14;

FIG. 16 is a longitudinal sectional view of a second embodiment of the applicator device of the present invention;

FIG. 17 is a horizontal sectional view of the applicator device shown in FIG. 16 taken along line 17-17 of FIG. 16;

FIG. 18 is a transverse sectional view taken along line 18-18 of FIG. 17;

FIG. 19 is a horizontal sectional view taken along line 19-19 of FIG. 16;

FIG. 20 is a horizontal sectional view of the applicator head of a third embodiment of the applicator device of the present invention;

FIG. 21 is a longitudinal sectional view of a modification of the applicator device as shown in FIG. 7;

FIG. 22 is a longitudinal sectional view of a modification of the applicator device as shown in FIG. 7;

FIG. 23 is a longitudinal sectional view of a modification of the applicator device as shown in FIG. 7;

FIG. 24 is a partial perspective view, in an enlarged scale, of the container handle shown included in the applicator device as shown in FIG. 1;

FIG. 25 is a perspective view of the connecting elements of the container and housing of a fourth embodiment of the applicator device of the present invention;

FIG. 26 is a partial bottom view of the container of the applicator device shown in FIG. 25, taken from line 26-26 of FIG. 25;

FIG. 27 is top partial view of the housing of the applicator device shown in FIG. 25, taken from line 27-27 of FIG. 25;

FIG. 28 is a partial longitudinal sectional view of the fourth embodiment of the present invention as depicted in FIG. 25;

FIG. 29 is a partial horizontal sectional view of the applicator device shown in FIG. 28, taken along line 29-29 of FIG. 28;

FIG. 30 is a transverse sectional view, in enlarged scale, taken along line 30-30 of FIG. 28;

FIG. 31 is a transverse sectional view, in enlarged scale, taken along line 31-31 of FIG. 28;

FIG. 32 is a detail sectional view, in enlarged scale taken along line 32-32 of FIG. 31;

FIG. 33 is a longitudinal sectional view, in enlarged scale, of a portion of the coupling included in the device shown in FIG. 28;

FIG. 34 is a partial perspective view, in an reduced scale, of the container shown included in the applicator device as shown in FIG. 25;

FIG. 35 is a partial longitudinal sectional view of a fifth embodiment of the applicator device of the present invention;

FIG. 36 is a horizontal sectional view of the applicator device shown in FIG. 35 taken along line 36-36 of FIG. 35;

FIG. 37 is a transverse sectional view, in an enlarged scale, taken along the line 37-37 of FIG. 36;

FIG. 38 is a longitudinal sectional view, in an enlarged scale, of a portion of the coupling included in the device shown in FIG. 35; and

FIG. 39 is a partial perspective view, in an reduced scale, of the container shown included in the applicator device as shown in FIG. 35;

### DETAILED DESCRIPTION OF THE PREFERRED EMBODIMENTS

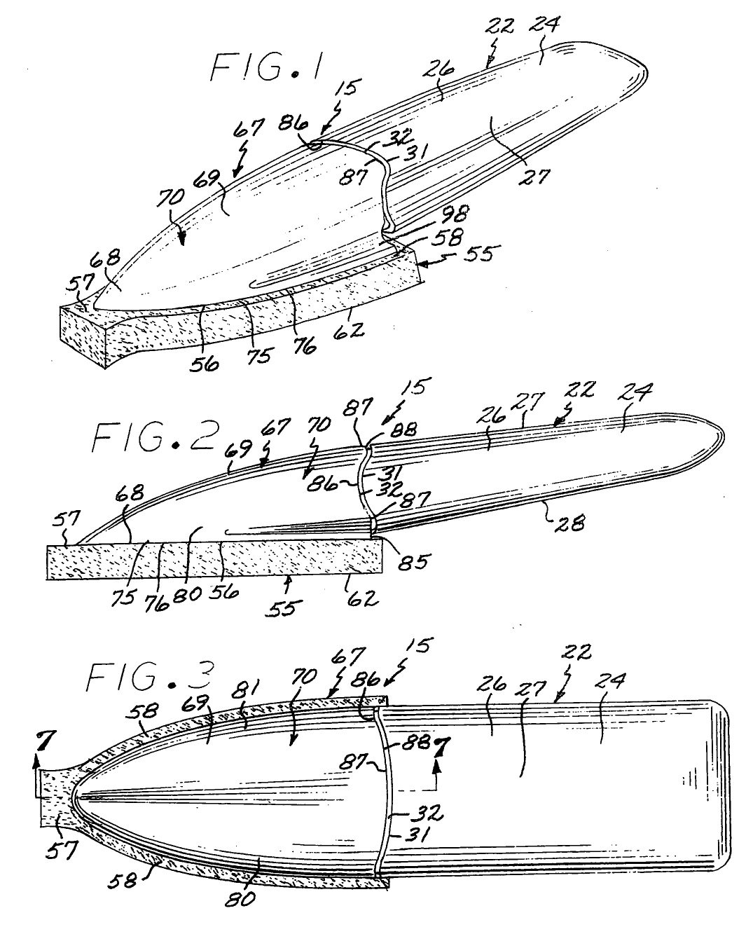

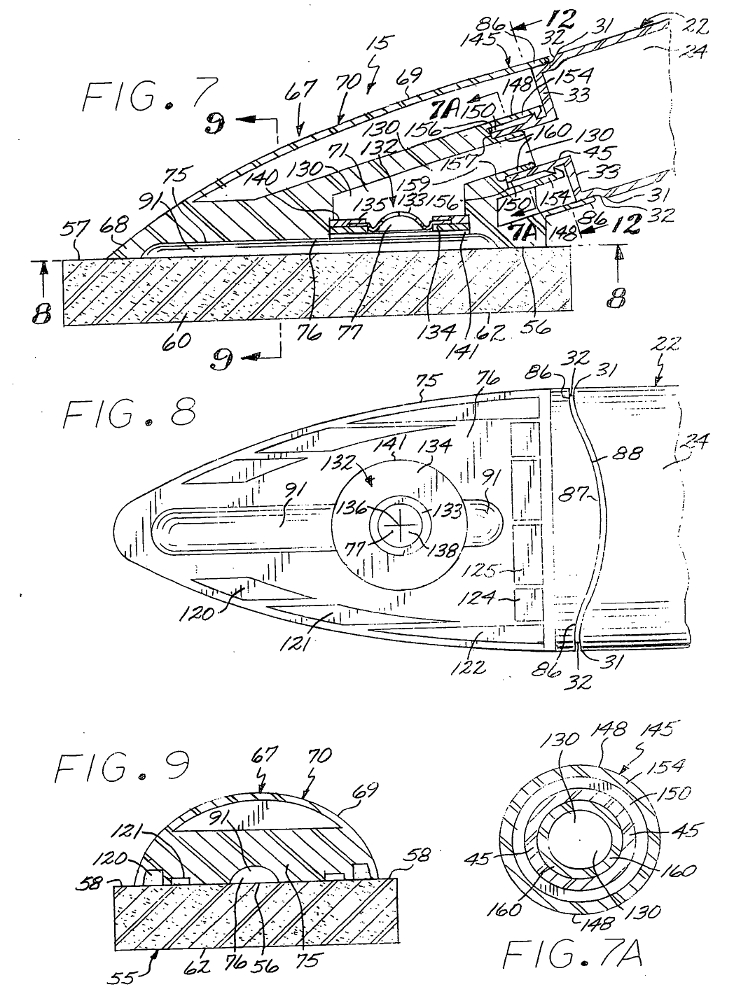

Referring to FIGS. 1-3, 7-9 and 25-26, the applicator device 15 of the present invention includes, generally, an applicator head 67, which includes a dispenser housing 70 and an applicator pad 55, and a container 22, which in a preferred embodiment both defines a container reservoir 24 for storing fluid and serves as an elongated handle. The housing applicator includes a 70 formed with top and side walls in a bottom surface, which as shown in the embodiment of FIGS. 7 and 8, may be formed by the downwardly facing surface of a distribution plate 75, which includes a distribution surface 76. The housing 70 further includes a flow chamber 71 which is bounded by a plurality of chamber walls and a container coupling assembly 145 including an inlet device 148 projecting rearwardly from the flow chamber for coupling with the container 22 to secure the container to the housing 70. The inlet device 148 may take on any convenient shape or form for transferring fluid therethrough to the flow chamber 71, and may include a coupling shell 154 and, as depicted in the embodiment of FIG. 7, a tubular inlet boss 160. A flow control device, generally designated 132, for metering the flow of fluid to the applicator pad 55 is interposed at some point along a fluid communication path 130 that extends from the container 22 and through the inlet device 148 and flow chamber 71 to the housing's bottom surface. The pad is mounted on one side to the bottom surface at an applicator pad attachment surface 56 by any appropriate affixation or bonding means as is well known in the art, and is further formed on the opposite side with a working surface 62 that is adaptable to engage the variety of surfaces found in an automobile interior.

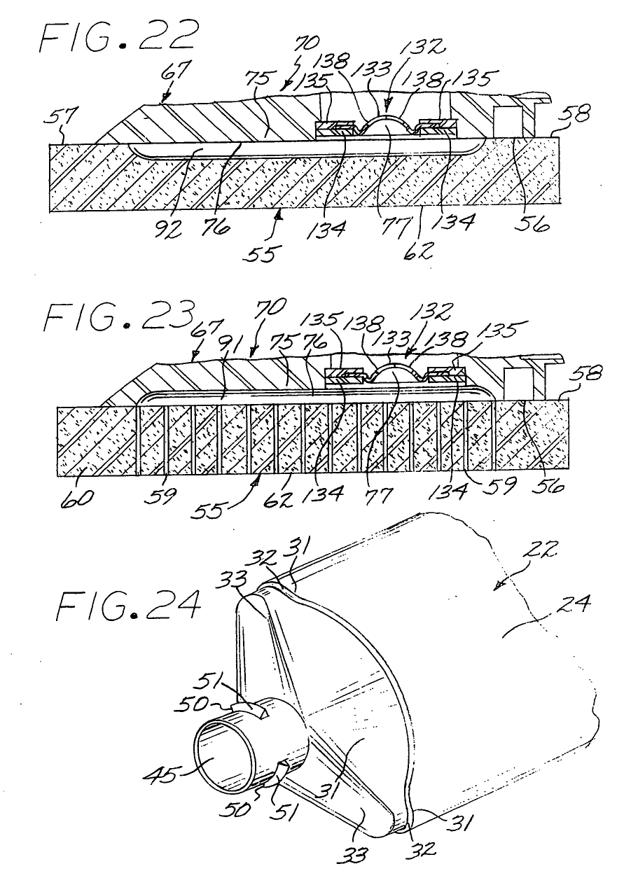

In the embodiment as depicted in FIG. 7 the bottom surface 78 of the distribution, the distribution plate 75, the distribution plate 75 is generally planar, however, it is contemplated that the plate may be formed with a curved or angled surface as required to be more adaptable to specific correspondingly shaped interior surfaces. The In such an embodiment, the distribution plate 75 is further formed with flow distribution capability, which, as shown in FIG. 7-8, may include at least one distribution opening 77 for communicating fluid from the flow chamber 71 to distribution surface 76, and least one longitudinal distribution channel 91. It is contemplated, however, that this distribution capability may take on any number of forms, such as, for example, the distribution plate 75 being formed with openings to pass the fluid therethrough, slits formed through the plate or in its distribution surface or a sieve type arrangement in the plate. While the embodiment of FIG. 8 depicts one such distribution channel 91, extending longitudinally on both sides of the flow control 132, which is shown as being disposed in the plate 75, it is also contemplated that a distribution plate 75 or distribution surface 76 may be formed with a plurality of such channels 91 extending across its longitudinal and lateral dimensions, or, as shown in FIG. 20, that a distribution surface 76″ may be formed with a plurality of channels 91′ extending from a central distribution manifold 96. It is further contemplated that the distribution channel or channels 91 (FIG. 8) may be formed with branches extending laterally and outwardly therefrom to facilitate the evenly dispersed flow of fluid across both the lateral and longitudinal area of the corresponding attachment surface 56 of the applicator pad 55.

As shown in FIG. 21, it is also contemplated that the attachment surface 56, on the top side of the applicator pad 55, may be formed with distribution channels 92 to further facilitate this flow. As shown in FIG. 22, the attachment surface 56 of the pad 55 may be formed with such channels 92 to independently or, in combination with the channels 91 (FIG. 21), serve as the distribution means. Further, while the distribution channels 91 and 92 are preferably formed in the distribution plate or confronting side of the pad, as will be apparent to those skilled in the art, such distribution may also be achieved by passages or channels formed in a plate or the like sandwiched into the interface between such plate 75 and pad 55.

The exemplary applicator pad 55 is of a semi-open cell foam construction and serves to receive fluid from its top side after the fluid has been directed by the flow chamber 71 to the housing bottom surface. The density of the pad 55 and the viscosity of the fluid is such as to restrict the rate at which the viscous fluid is dispensed therethrough. In practice, after the fluid travels through the flow chamber and is deposited on the attachment surface 56, a portion of the fluid will flow through the local area of the pad. The remainder of the deposited fluid will pool on the attachment surface 56 and then will be further distributed longitudinally and laterally to other desired portions of the pad's. In the embodiment of FIGS. 7-9, the fluid will travel along the distribution channel 91 to be distributed longitudinally along the center of the pad 55, and laterally through distribution branches if present.

With reference to the preferred embodiment of FIG. 23, in order to facilitate the transfer of fluid through the applicator pad 55 to specific strategic locations on the working surface 62, the pad 55 may be formed with through channels 59 arrayed thereabout and extending from the attachment surface 56 to the working surface 62 to facilitate even distribution to the working surface 62. It is also contemplated that pin holes (not shown) punched in the attachment surface 56 may be situated thereon to promote absorption and flow through the pad 55 at specific desired locations, or that the area of the pad not incorporating a distribution channel may also incorporate through channels 59 for passing fluid from the attachment surface 56 to the working surface 62. Additionally, it is also contemplated that, in order to promote a more rapid transfer of fluid through the pad 55 to desired portions of the working surface 62, such as, for example, the lateral extremities of the pad, these desired portions may be formed with pre-cut indentations defining a stepped down transverse cross sectional depth or may be formed from a more porous material than is found in the remainder of the pad 55.

The applicator pad 55 may take any convenient shape, size and dimensions that are adapted to provide a lower working surface 62 for engagement with the variously dimensioned and shaped interior surfaces of an automobile. As shown in FIGS. 1 and 2, the applicator pad 55 is configured with the attachment surface 56 to be attached to the solid portions of the housing bottom surface, which may be partially defined at the bottom edges of the walls of the flow chamber 71 or the distribution surface 76 of the distribution plate 75, at an interface therebetween by one of the many suitable bonding agents or other affixation means known in the art. To this end, it is contemplated that the distribution surface 76 in the embodiment of FIGS. 7-9 may be formed with a smooth and solid surface, or may be formed with any appropriate surface pattern, such as a grid or parallel ridges, to provide surface area for bonding the attachment surface 56 of the pad thereto. As shown in FIG. 8, it is also contemplated that the distribution surface 76 may be conveniently formed along its lateral opposite edges with downwardly opening shallow, blind cavities 120, 121 and 122 which act as lightening holes. The rear edge of the distribution surface 76 may also be formed with a row of laterally projecting downwardly opening lightening cavities 124 and 125. The contours of these cavities, which can also take on any convenient shape, dimension and location, cooperate in defining the distribution surface 76 to which the pad 55 is mounted.

The pad 55 is conveniently constructed in the form of semi-open cell polymer sponge like material, which can be either formed by injection molding or cut from a stock of foam such as is well known in the art as being suitable for this purpose. However, while the viscosity of the fluid will influence its rate of flow through the pad 55, it is contemplated that the pad may be formed of any material conducive to providing a desired level of resistance to prevent rapid fluid transfer therethrough to the working surface 62, and that the viscosity and flow characteristics of the fluid, and the requirements of the chosen application, will influence the selection of this material. Therefore, the material composition, shape and dimensions of the pad 55 may be varied to suit a desired application or to work most effectively with the formulation and viscosity of the chosen treatment fluid. For example, it is contemplated that the applicator pad 55 may be formed with semi-open, open or closed cell foam, or with fibers having similar characteristics, or with bristles, such as those found in a brush, or with a porous flow control screen or plate or any other suitable material or structure for passing fluid therethrough to the working surface 62.

While not essential to the present invention, the pad 55 is preferably formed oversized relative to the plan view of the housing bottom surface to project laterally outwardly on opposite sides to form respective peripheral skirts 58. Further, as shown in FIGS. 1 and 2, it is contemplated that the pad 55 may also project forwardly beyond the distal portion of the housing 70 which defines a nose 68, with this forwardly projecting portion defining a blunt flexible applicator finger 57 to facilitate the longitudinal forward reach of the applicator pad. It is also contemplated that the pad 55 may correspond generally in cross-sectional shape and surface area to that of the housing bottom surface, or that the lateral edges of the pad's outer perimeter may angle downwardly and outwardly to culminate in and define an applicator pad working surface 62 of similar shape to that of the attachment surface 56 and housing bottom surface, but correspondingly greater in surface area.

Turning now to the construction of the housing 70, it may take any convenient shape or form, having, for example, an oval, semi-circular or triangular shape, and in a preferred embodiment, is conveniently configured in a somewhat half-bullet nose shape taking on the general appearance of a flat iron, as shown for example in FIG. 3. It may be formed of any convenient and suitable material, but is preferably formed from polypropylene or of any appropriate molded high density plastic, as are known in the art. The housing 70 has a shell 69 that may include a pair of laterally spaced apart side walls, 80 and 81, and generally defines a somewhat cylindrical transverse cross section. The shell 69 tapers rearwardly and upwardly from the somewhat pointed forwardly disposed nose 68 (see e.g. FIG. 2), while angling rearwardly and laterally outwardly to form, at the opposite end from the nose 68, a cowling 86. A housing rear wall extends downwardly from the bottom edge of the cowling 86. For receiving the container 22 in the housing 70, the housing may be formed rearwardly with the coupling assembly 145, which may include the somewhat oval in transverse cross section cowling 86 disposed about the inlet device 148, as shown in FIG. 7. Thus, as is evident from continued reference to, for example, FIGS. 2 and 7, the shape of the dispenser housing 70 and applicator pad 55 permits the applicator device 15 to treat many hard to reach surfaces that are not easily reachable by other applicator devices, such as those immediately adjacent to the intersection of an automobile's dashboard with its windshield.

It is contemplated that, as shown in FIGS. 1, 5 and 6, the respective side walls 80 and 81 may be formed in the vicinity of their lower proximal extremities with respective laterally projecting side wings, 98 and 99, extending outwardly from the dispenser housing 70 to define a housing bottom surface of relatively greater surface area for affixation of a correspondingly greater in surface area applicator pad 55 thereto. The design of this embodiment lends greater stability to the applicator pad over an increased surface area, and further facilitates the sustained and inwardly directed application of force by way of the working surface 62 as it is in contact with the automobile interior. While it is contemplated that the user will generally gain favorable purchase of the applicator device 15 by grasping the container 22 as a handle, the side wings 98 and 99 and/or side walls 80 and 81 may provide respective convenient finger grooves or pads that permit the user to grasp the device by the housing 70 and cowling 86 when he or she desires to exert a greater and more focused degree of inwardly directed force to a given treatment surface.

With continued focus on the structure of the housing 70, the coupling assembly 145 may include the rearward portion of the dispenser housing 70 and cowling 86, and is adapted to receive the container 22 therein. As shown, for example, in FIG. 24, to be received in the coupling assembly 145, the container may include an end wall 31 and a yoke 33 centrally formed with an outwardly extending neck 45. The coupling assembly 145 may be adapted to receive the neck 45 and yoke 33 while mating with complemental surfaces in the end wall 31 of the container. In one embodiment, as shown in FIG. 7, the inlet device 148 of the coupling assembly 145 projects upwardly and rearwardly relative to the housing bottom surface to form an axis at about 20° to the working surface 62 formed by the underside of the applicator pad 55. It is contemplated, however, that this angle may be increased or decreased to optimally promote the flow of fluid from the container 22 through the flow chamber 71 to the applicator pad 55, and may depend on considerations such as the desired application and treatment surfaces and the flow characteristics of the fluid.

In one embodiment, as shown in FIGS. 7 and 7a, the inlet device 148 may further include the inlet boss 160 extending from flow chamber 71, and may also include a coupling shell 154 disposed concentrically about the inlet boss 160 to form therebetween a rearwardly opening annular cavity 150 for receipt of the neck 45 projecting forwardly from container 22 (see also FIG. 24). The inlet boss 160 is specifically dimensioned to be received within the neck 45 with the neck disposed thereabout in a friction fit relationship. While an annular configuration has been depicted for the cavity 150 to receive the neck, it is contemplated that the cavity 150 may be formed in any convenient and appropriate shape for receipt therein of a corresponding in shape container neck 45. As shown in the embodiment of FIGS. 7, 14 and 15, the inlet device is also formed with a coupling wall 156 that defines an outwardly facing neck abutment surface 157 such that the distal extent of the neck 45 is abutted thereagainst when the neck is received in the annular cavity 150 and telescoped over the inlet boss 160. A central opening 159 (FIG. 7) formed in the coupling wall 156 permits the flow of fluid therethrough to the adjacent flow chamber 71. To operate in conjunction with the structure of the neck 45 to releasably connect the housing 70 to the container 22, as shown in FIGS. 7 and 14-15, the coupling shell 154 of the inlet device 148 is further formed at its distal extremity with a plurality of inwardly projecting lugs 162, which are arrayed thereabout and spaced apart to define respective clearance slots 165 therebetween. For example, in the embodiment depicted in FIGS. 12 and 13, three such lugs 162 are spaced annularly equidistantly apart to define three corresponding clearance slots 165 therebetween.

With the container 22 received in the housing 70, the neck 45, inlet device 148, flow chamber 71, and distribution plate 75 and distribution opening 77, if included, cooperate to define fluid communication path 130 therebetween for flow of fluid from the container 22 to the applicator pad 55. Positioned at some point along this fluid communication path 130, a flow control 132 functions to control the flow of fluid therethrough. For example, the flow control may be located in the inlet device 148 or flow chamber 71 in the vicinity of the bottle neck, or may be situated in the distribution plate 75 if present.

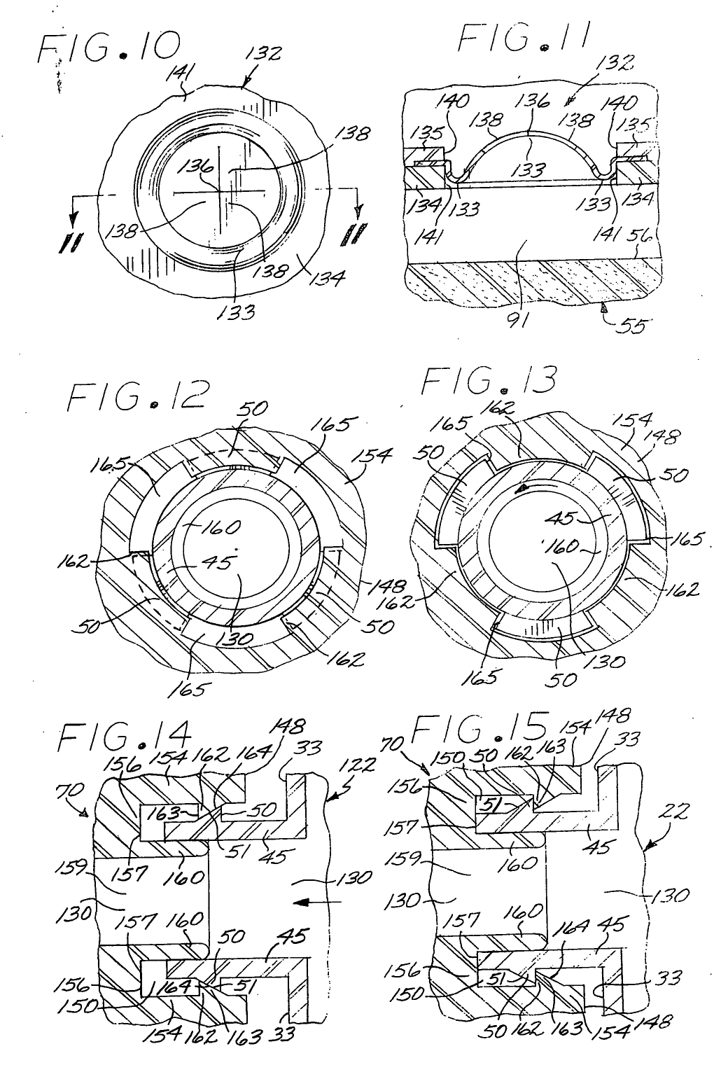

In the latter example, as shown in FIGS. 7 and 11, the distribution plate 75 may be further formed with a through bore 140 for communicating with the under side thereof. Such bore 140 is counterbored from the bottom at counterbore 141 for nesting there up into the flow control 132. While this nesting may be accomplished by a variety of suitable constructions, in the preferred embodiment as shown on FIGS. 10-11, the flow control 132 includes a pair of mounting rings, 134 and 135, received telescopically in the counterbore 141, which mount centrally therein a control valve 133. While the construction and material composition of the valve 133 may be varied depending on the viscosity of the treatment fluid and the desired flow characteristics for a given application, in the preferred embodiment depicted in FIG. 10-11, the control valve 133 is a one way flow valve in the form of a flexible polymer sheet configured with a dome having a cruciform slit 136 therein (FIG. 10) to form diametrical slits oriented at 90° to one another to form triangular leaves 138. Upon application of fluid pressure to the top side thereof, radially inward points of these leaves 138 are flexed downwardly and outwardly to cooperate in forming an opening for downward flow of fluid therethrough into the distribution channel 91 and onto the applicator pad attachment surface 56. Upon release of such top side fluid pressure, further flow of fluid through the opening in the valve 133 will be prevented as the leaves 138 return to their original closed configuration.

While a one way valve embodiment has been described, the flow control 132 may take on a variety of forms known in the art, for example a porous disc, duck bill or flapper valve, membrane, other types of valves or any other suitable means for metering the flow of fluid therethrough to a predetermined rate. Also, in the embodiment of FIGS. 7-8, the flow control 132 is disposed in the distribution plate 75, however, it may be located at any other point along the fluid communication path 130 extending from the container 22 to the applicator pad 55 so long as it functions to control the flow of fluid therethrough. For example, the flow control 132 may also be disposed within the flow chamber 71 or the inlet device 148. It is also contemplated that the flow control 132 may be located at the distal extremity of the neck 45, and take the form of any appropriate squeeze bottle type flow control or opening known in the art. Further, the viscosity of the fluid may also influence the chosen construction of the flow control 132. For example, it is known in the art that lower viscosity fluids are more likely to be inhibited from flowing through a one way flow type valve than those fluids having a higher viscosity. Thus, it is contemplated that the specific construction of the flow control 132 may also vary depending on the material composition of the chosen treatment fluid to be dispensed therethrough, as is known in the art.

Focusing now on the container 22, as shown in FIGS. 1 and 2, it includes a dorsal wall 26, a ventral wall 28 and a end wall 31. The container 22 may be multi-purpose in that the distended, self-supporting flexible walls cooperate to define an elongated, somewhat oval in transverse cross-section handle, by which the user may gain favorable purchase of the applicator device 15, while also defining a fluid reservoir 24 containing a supply of cleaning or protecting fluid. In a preferred embodiment as shown in FIGS. 1-3 and 24, the container 22 may take the form of a squeeze bottle formed of a durable yet resilient plastic to form walls to, in their unflexed configuration, maintain the shape and outward dimensions, but compressible inwardly by squeezing to reduce the interior volume to elevate the interior pressure to drive the fluid out into the flow path and distribution network. Being self-supporting, upon release of the squeezing force, such walls will distend to their unflexed positions, thereby drawing a partial vacuum in the reservoir, providing for atmospheric pressure to force air into the reservoir to cooperate with the residual fluid to occupy the full volume thereof. Therefore, it is contemplated that the container 22 may be formed from a multiplicity of appropriate materials encompassing a wide range of durability and resiliency, as are known in the art. For example, polypropylene, polyethylene, polyvinylchloride and the like have proven to be suitable materials for the container 22. The material composition of the container 22 is sufficiently rigid so that it may serve also as a handle by which a user may grasp the applicator device 15 and exert adequate inwardly directed force to focus and control the application of treatment fluid to a desired automobile interior surface.

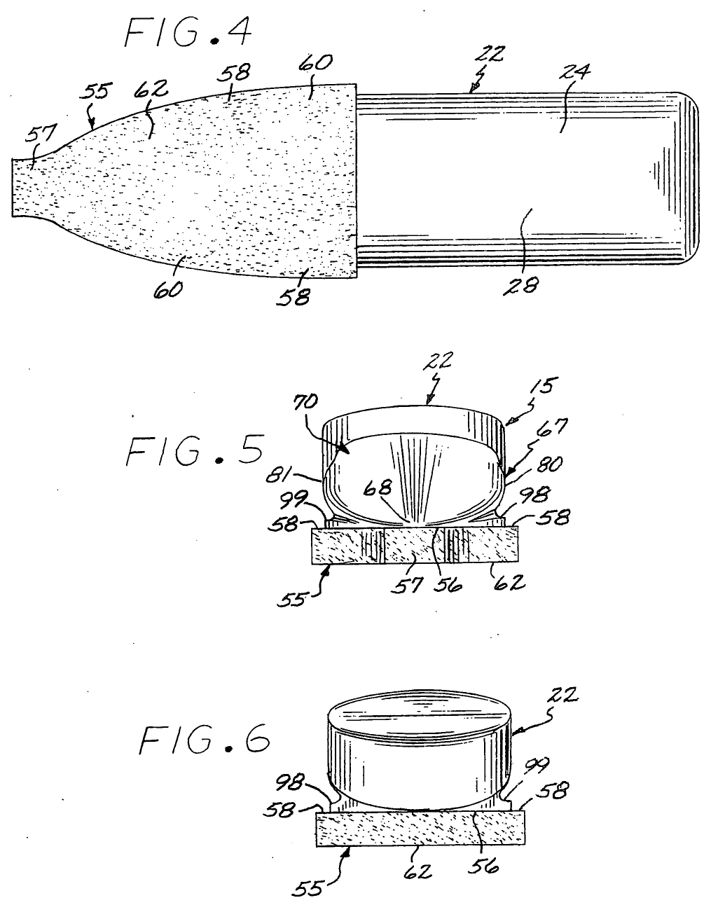

It is contemplated that the squeeze bottle container 22 depicted in the preferred embodiment of FIGS. 1-4 may be disposable and replaceable, containing any number of a variety of appropriate treatment fluids for application to an automobile's interior surfaces. The user may detach the squeeze bottle container 22 from its complementally mating applicator head 67 and discard it when it has exhausted its supply of fluid, while subsequently replacing the discarded bottle with a new and filled bottle. However, it is also contemplated that the squeeze bottle container 22 may be refillable by way of an outwardly and upwardly extending filling stem (not shown) projecting from the vicinity of the rear extremity of the dorsal wall 26. It is further contemplated that such a filling stem may include a snap on containment cap, a screw top or hinged construction or any other appropriate securement means (not shown) to prevent the escape of fluid from the reservoir 24.

The exterior surface of the container 22 need not be specifically ergonomically adapted, however, as shown in the preferred embodiment of FIGS. 1 and 2, at least the dorsal wall 26 may be shaped and adapted to correspond to the natural curve of a typical user's palm when he or she is grasping the container 22 as a handle, while the ventral wall 28 may be similarly shaped and oppositely disposed. In plan view, as shown in a preferred embodiment of FIG. 3, the convex dorsal wall 26 curves gradually outwardly and downwardly to define a palm pad 27 for complemental receipt in the correspondingly concavely curved palm of the user when his or her hand is in a grasping posture. This palm pad provides a pressure surface facing in one direction by which the user may grasp the applicator to exert an appropriate amount of force in the opposite direction for applying treatment fluid to a desired surface. It is further contemplated that other ergonomic features may be incorporated into the container 22 design, to include, for instance, finger grooves (not shown) for receipt of the user's fingers therein.

With focus now on the connection of the container 22 to the dispenser housing 70, as shown, for example, in FIGS. 1-3, 7-8 and 25, the cowling 86 terminates in its rear edge in a scallop configured on its top and bottom sides with rearwardly projecting curved tongues 87 terminating in respective rearward edges 88. Referring to the preferred container 22, as shown, for example, in FIG. 24, a contoured groove is formed about the periphery of the end container wall 31 to define a forwardly facing contoured shoulder 32 curved on its opposite sides to receive in a nesting relationship the respective tongues 87. Also, as exemplified in FIG. 24, the end wall 31 of the container 22 may include a yoke 33 that extends from the lower extent of the shoulder 32 to define the portion of the container 22 that is received within the coupling assembly 145 of the housing 70. The yoke 33 is preferably centrally formed with the outwardly projecting neck 45 to be received in cavity 150 of the inlet device 148 (see e.g. FIGS. 14-15). The neck 45 may take any convenient corresponding shape to that of the cavity 150 for complemental receipt therein, and in one preferred embodiment as shown in FIG. 24, is internally hollowed along its length and cylindrical in shape. It is also contemplated that a bottle cap (not shown), which may take on a multiplicity of structures known in the art, may be releasably secured over the proximal end of the neck 45 to seal against the unwanted flow or evaporation of fluid from the container reservoir 24. A user may remove and discard this cap before mating the container 22 with the dispenser housing 70, or may retain it to be placed back on the neck 45 if the container 22 is removed from the applicator head 67 for storage between applications.

With continued reference to the embodiment of FIGS. 24, 7 and 12-15, to enable mounting and locking of the container 22 into the inlet device 148 of the dispenser housing 70, the neck 45 is formed with a plurality of radially outwardly projecting locking studs 50. Such studs 50 are annularly arrayed about the neck 45 and spaced apart and sized to snapingly register behind corresponding lugs 162 (see FIGS. 14-15) in the inlet device 148 and to fit axially through the clearance slots 165 (see FIGS. 12-13). The studs 50 are further configured at their respective free extremities with outwardly and rearwardly angled cam surfaces 51. As shown in the embodiment of FIGS. 12 and 13, the neck may be formed with three such studs 50 for coupling with three corresponding lugs 162 on the coupling shell 154, which are arrayed equidistant thereabout and spaced annularly apart by a distance to define respective clearance slots 165 therebetween, and to receive axially, in clearing relationship, the respective studs 50. As shown in FIGS. 14 and 15, such lugs 162 are configured with radially out turned teeth 163 defining inwardly and forwardly angled, outwardly facing cam surfaces 164 configured to slidingly engage the cam surfaces 51 of the studs 50 for axial shifting relative thereto and flexing to provide for axial travel sufficient to register the studs 50 behind the lugs 162 in locking relationship as shown in FIG. 15. So configured, the neck 45 will be received in the annular cavity 150 and over the inlet boss 160 (if present) such that, with the studs 50 engaged securely behind respective lugs 162, the distal portion of the neck 45 will be seated against neck abutment surface 157, as is shown in FIG. 15, and the neck 45 will be securely seated in inlet device 148 in a close fit relationship to provide a fluid tight sealing engagement between the container 22 and the housing 70. Thus, with the rearward edges 88 of the cowling tongues 87 nested against the forwardly facing shoulder 32 of the end wall 31, the neck yoke 33 received in the coupling assembly 145, the neck 45 seated against the abutment surface 157 and received over the inlet boss 160 (if present) and the studs 50 registered securely behind respective lugs 162, the container 22 will be securely registered within the housing to hold its rotary position therein.

To release the container 22 from the dispenser housing 70 and its coupling assembly 145, either the cowling 86 and/or cowling tongues 87 (see e.g. FIG. 2) or the yoke 33 and/or container end wall 31 (see e.g. FIG. -24), or all of these elements, may be constructed of a material sufficiently flexible to permit sufficient limited axial rotation of the container 22 and the cowling 86 relative to one another to disengage the complemental mating of the forwardly facing shoulder 32 of the container 22 and the rearward edges 88 of the curved cowling tongues 87. This simultaneously rotates the neck 45 within the coupling shell 154. For example, the neck 45 may be rotated from the position shown in FIG. 12, with the studs 50 snapingly engaged behind corresponding lugs 162, until the locking studs 50 are aligned with respective clearance slots 165, as shown in FIG. 13. The user may then withdraw the studs 50 axially through the slots 165 to effectuate a separation of the neck 45 from the inlet device 148. It is also contemplated that, to disengage the container 22 from the housing 70, the cowling 86 and container 22 may be manufactured such that, when the yoke 33 is received in the cowling 86 and the cowling tongues 87 are aligned with the container shoulder 32, there is sufficient clearance between the shoulder and the tongues and the yoke and the cowling to permit limited axial rotation of the container 22 relative to the housing 70.

While a snap lock connection has been described, it is contemplated that any appropriate connection means, such as a bayonet fit, threaded engagement or a clamp type connection, may be employed in the coupling assembly 145 to facilitate coupling of the container 22 to the dispenser housing 70. As for example shown in FIGS. 37-39, the coupling shell 154 may be configured with a peripheral connector bead section while the neck 45 is formed with an exterior conically shaped flange for snapping behind this connector bead section. It is also contemplated that female threading in the coupling shell 154 may receive male threads formed on the neck 45, or that male threads on the periphery of the inlet boss 160 (if present) may be received in female threading on the interior of the neck 45. Additionally, while the container 22 has been shown as including a projecting tubular neck 45 for receipt in the coupling assembly 145 of the housing 70, it will be appreciated by those skilled in the art that the term neck is intended to include any opening in the container, including a recessed tubular element, it only being important that the construction of the neck permit complemental mating of the housing 70 and the container 22.

In operation, it will be appreciated that the applicator of the present invention will typically be sold at a retail level in a package including the applicator head 67 and container 22, possibly along with one or two replacement containers. The replacement containers will typically be closed by a cap (not shown) releasably connected to the container's neck 45 by any suitable means known in the art. To assemble the applicator device 15, the user will mount a chosen container 22 in the applicator head 67 by generally inserting the yoke 33 and end wall 31 of the container 22 into the coupling assembly 145 of the housing 70. More specifically, the snap lock construction included in the coupling assembly 145 of the embodiment shown in FIGS. 12-15 permits the user to seat the container neck 45 in the inlet device 148 in a close fit, fluid tight sealing relationship, by inwardly advancing the neck 45 through the cavity 150 within the coupling shell 154 and over the inlet boss 160 until the neck studs 50 are snapingly engaged behind respective lugs 162 and the distal extent of the neck 45 is seated against the neck abutment surface 157. This serves to align the mating curvilinear rearward edges 88 of the cowling tongues 87 with the forwardly facing shoulder 32 of the end wall 31 as shown for example in FIG. 8, while the yoke 33 and end wall 31 of the container are seated in the coupling assembly 145 and the neck 45 is received in the inlet device 148.

When the user undertakes to use the applicator, he or she will grasp the container 22, hold the head 67 down, and either shake such container or exert inwardly directed compressive force on the walls thereof to reduce the volume of the reservoir, applying pressure to the applicator fluid therein to drive such fluid downwardly along fluid communication path 130 through the boss 160 (if present as shown in FIG. 7) and downwardly into the flow chamber 71. In this regard, it will be appreciated that by pointing the housing 70 downwardly, the fluid will travel into the flow chamber 71 and along the communication path 130, which will apply pressure to the flow control valve 133. As in the embodiment of FIG. 7, with the flow chamber 71 and inlet boss 160 filled, by compressing the walls of such container 22 and reducing the volume therein, pressure will be applied to the fluid in the flow chamber 71, thus tending to force it through control valve 133 (FIGS. 10-11). As further pressure is applied thereto, the valve's domed shape will be deflected downwardly in the center, thus flaring the proximate corners of the leaves 138 downwardly, thereby opening the slits 136 and providing for a flow of treatment fluid downwardly through the distribution plate 75 and distribution surface 76 to the applicator pad attachment surface 56. A portion of the deposited fluid will begin to flow through the applicator pad 55, while the remaining fluid begins to flow through the channel 91 to travel forwardly and rewardly therein, as shown in FIGS. 7-8, so that fluid is distributed across the lateral and/or longitudinal dimensions of the applicator pad 55 for passage therethrough to the working surface 62.

The user will then grasp the container handle 22 to gain favorable purchase of the applicator 15 and may move the handle as desired to pass the head 67 of the applicator across the surface to be treated, thus applying fluid reaching the underside working surface 62 to the treatment surface. The handle container 22 serves to extend the reach of the applicator 15, and in practice, the applicator head 67 is about 4 inches long and the container 22 about 6 inches long to provide an overall axial reach of some 10 inches. By grasping the container 22 and thrusting the tapered head forwardly, the operator may conveniently access, for instance, the surface of automobile dashboard, even forwardly into the triangular volume formed between the generally horizontally rearwardly projecting dashboard surface and interior of the rearwardly upwardly sloped windshield. If desirable, when the interior surface of the door or like areas are being treated, the user may conveniently grasp the dispenser housing 70 from the top side thereof, applying the palm of his or her hand to the domed surface thereof, to thus there apply more direct perpendicular forces against the applicator pad 55 to increase the application force on the working surface 62 and the polishing and application effect thereof.

It will be appreciated that the forwardly projecting finger 57 (FIG. 1) of such applicator pad 55 and/or the peripheral skirts 58 will compress from the bottom and top sides to conform to the contours of the areas being accessed to thus allow the user to reach even the most narrow area between, for instance, the windshield and dashboard surface. Additionally, when the user engages the working surface 62 of the pad 55 with a desired treatment surface, the pad 55 is flexible to flex and cooperate with working surface 62 to conform to the shape and curvature of the chosen treatment surface. This will permit the user to evenly spread the desired fluid onto the treatment surface by applying a substantially even pressure across the length of the working surface 62. It will be appreciated that further downward pressure on the applicator head 67 will facilitate the tendency to force the liquid through such pad 55 to the working surface 62 and to the surface being treated. When the initial charge of fluid dispensed has been depleted, the user may thereupon squeeze the container 22 or otherwise again repeat the above described sequence.

When the procedure is completed, the user may easily disconnect the container 22 from the dispenser housing 70 and coupling assembly 145 by twisting the container 22 to rotate container end wall 31 within the cowling 86. The flexibility of the cowling 86, curved tongues 87, yoke 33 and/or end wall 31 will permit limited axial rotation to skew the alignment between the end wall 31 of the container 22 and the curved tongues 87 of the cowling 86, thereby disengaging the forwardly facing shoulder 32 of the container 22 from the rearward edges 88 of the tongues 87. In the embodiment of FIG. 7, this simultaneously permits the user to similarly rotate the neck 45 slightly within the coupling shell 154 and cavity 150 from the position shown in FIG. 12, with the studs 50 snapingly engaged behind respective lugs 162, until the locking studs 50 are aligned with respective clearance slots 165, as shown in FIG. 13. The user may then withdraw the studs 50 through the slots 165 to effectuate a separation of the container 22 from the housing 70.

A cap (not shown) may then be replaced on the neck 45 of the container 22 to be stored until the next use, and, if desirable, the applicator pad 55 may be cleaned or washed in a cleaning fluid, such as tap water. The container 22 and applicator head 67 may then be readily assembled for the next usage, or when the fluid in such container becomes diminished, the container 22 may be discarded and a new replacement container 22, already charged with a desired fluid, may be selected and secured in the dispenser housing 70 as set forth above. It is contemplated that the user may replace the depleted container with another of the same type container for treatment of a similar surface, or may select a different container having appropriate treatment fluid for application to a different treatment surface.

Turning now to an alternate embodiment as depicted in FIGS. 16-19, it is also contemplated that the housing bottom surface may incorporate a distribution plate 75′ formed with a plurality of through flow openings 100 arrayed across the longitudinal and lateral extent thereof. As shown in FIG. 16, in this preferred embodiment, a housing 70′ is formed with a flow chamber 71′. The flow chamber 71′ may also include a multiple chamber internal construction, being divided into a plurality of chambers, for example two, or, in the embodiment depicted in FIG. 8, a central introduction chamber 72 may be disposed between a pair of flanking chambers 73. However, it is also contemplated that the fluid may pass through the flow chamber 71′ to a distribution manifold (not shown), which in turn distributes fluid to a plurality of transfer channels for distributing the fluid across the dimensions of the attachment surface 56 and through the applicator pad 55 to its working surface 62.

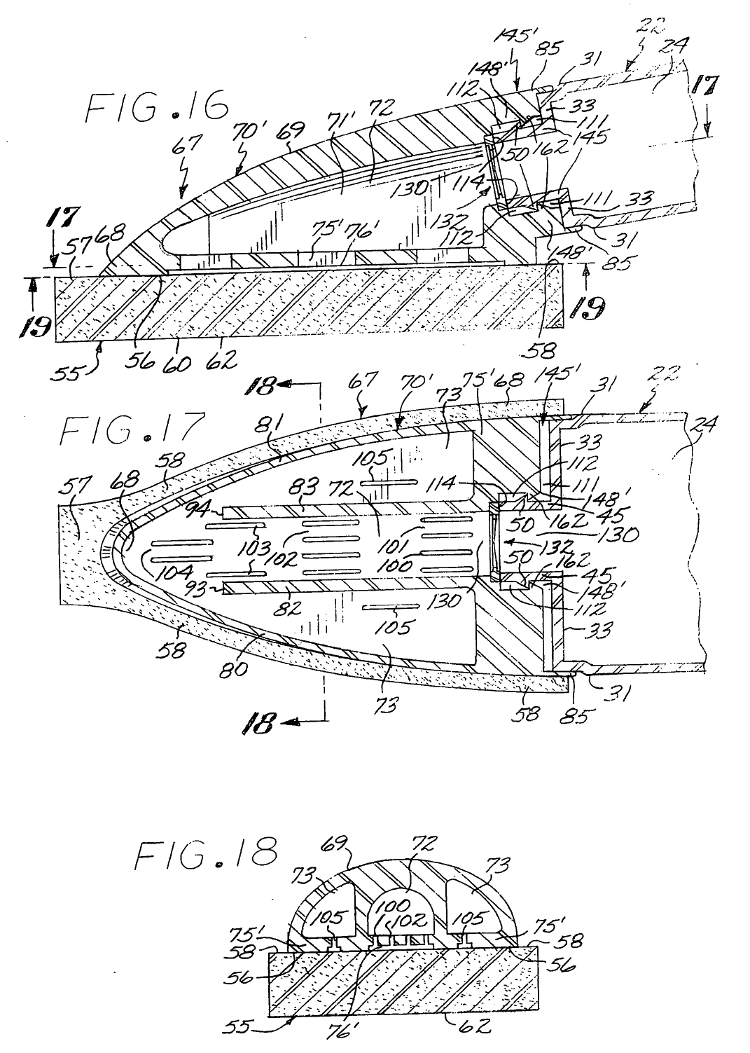

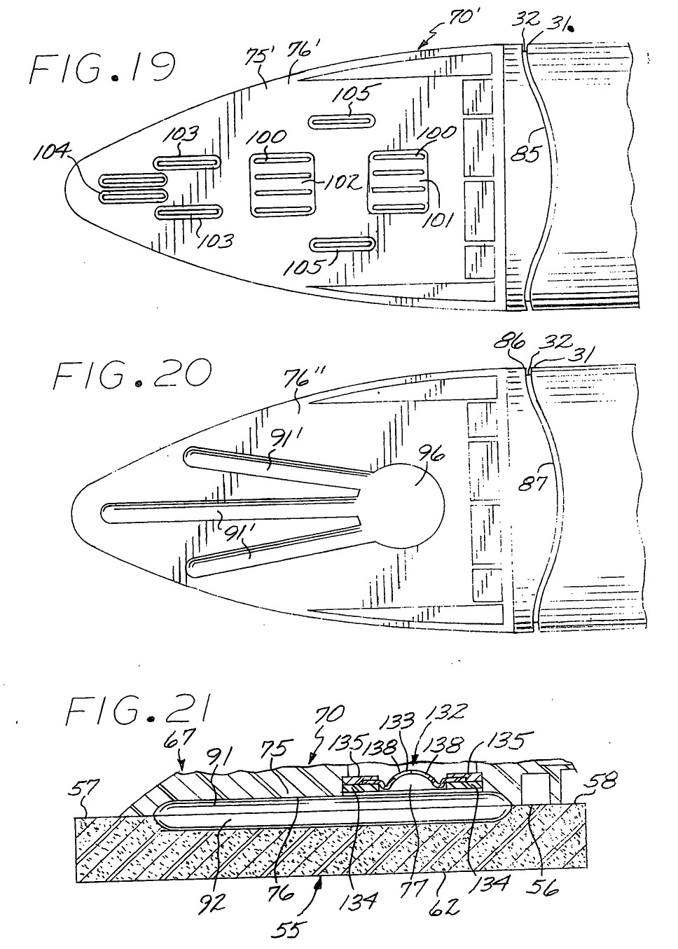

With continued reference to the preferred embodiment depicted in FIG. 17, in a tripartite multiple chamber embodiment, the chamber 71′ may be configured with a plurality of chamber walls and with a pair of elongated laterally spaced apart ribs, 82 and 83. In this embodiment, the housing 70′ further includes a housing rear wall 85, and the ribs, 82 and 83, emanate from the rear wall 85, projecting forwardly to form a centrally disposed introduction chamber 72 and to terminate at their respective forward extremities in respective outlet edges 93 and 94 spaced rearwardly of the laterally disposed converging sidewalls 80 and 81 of the housing 70′. Within the flow chamber 71′, these ribs, 82 and 83, not only define the introduction chamber 72, but their lateral edges also define the inner walls of a pair of laterally spaced apart flanking chambers 73 having the introduction chamber 72 disposed therebetween, with the respective flow chamber walls defining the outer walls of the flanking chambers. The top surface of the distribution plate 75′ defines the bottom surface of the flow chamber 71′ and any other chambers included therein.

In the embodiment as shown in FIGS. 16-17, the introduction chamber 72 angles downwardly and forwardly from the proximal extremity of the housing 70′ to terminate near the distal extremity, but may extend in any appropriate angle or configuration to facilitate the desired distribution of fluid through various locations in the distribution plate 75′. While fluid distribution to the distribution plate 75′ will generally be influenced by the pressure created by inwardly directed compressive forces on the walls of the container, the longitudinal alignment of the introduction chamber 72 may also influence the flow path of the fluid to the distribution plate 75′. For example, a greater downward and forward angling introduction chamber 72 permits the fluid to flow more to the distal extremity of the housing 70′, while a lesser downward and forward angling permits the fluid to flow more predominantly to the vicinity of the proximal extremity.

With reference to the embodiment of FIG. 17 wherein the distribution plate 75′ is formed with selected arrays of flow openings 100, these openings are strategically placed to distribute a metered and relatively predictable amount of treatment fluid therethrough to the applicator pad 55. In FIGS. 17 and 19, the openings appear as elongated slots 100, but may take any convenient shape or dimension to accommodate the material characteristics of the product being dispensed or the contours of the desired treatment surface. For instance, more viscous fluids will require larger openings. Also, smaller, hard to reach surfaces may require that there be more product near the distal extremity of the housing 70′ for dispensation through the applicator pad 55 near the distal tip thereof, thereby necessitating relatively more or larger openings 100 in the vicinity of the distal extremity of the distribution plate 75′ than near the proximal extremity.

With continued reference to the embodiment as shown in FIG. 17, a plurality of slots, generally designated 100, are arrayed in the distribution plate 75′ and may be grouped in a first and second set of longitudinally spaced apart slots, 101 and 102 respectively, which are generally situated in the introduction chamber 72 near the central region of the dispenser housing 70′. As will be appreciated by those skilled in the art, such relatively closely spaced and clustered slots, as shown in FIG. 17, are so configured to provide for the dispensation of a relatively robust quantity of fluid located generally centrally over the applicator pad 55 in the wider area thereof so as to afford a relatively robust quantity of dispensed fluid in that wide area for distribution and application to the desired interior automobile surface. It is contemplated that in one preferred configuration, these slots may be approximately 1/16″ wide and ⅜″ long for effective use in conjunction with a variety of commercially available multi-purpose protectant fluids. Other suitable treatment fluids may require appropriate adjustment in the dimensions of the slots 100 for optimal flow characteristics therethrough based on the material composition of the selected fluid. With ongoing reference to the embodiment of FIG. 17, disposed centrally near the distal extremity of the housing 70′, toward the forward extremity of the distribution plate 75′, are less densely clustered through slots 100 with one pair 103 being laterally spaced about ⅜″ apart and a forwardly disposed pair 104 spaced laterally at about ¼″ apart to thus provide for a less robust distribution of fluid at the more narrow forwardly disposed locations of the distribution plate 75′ near its distal extremity. As further shown in the embodiment of FIG. 17, spaced generally centrally in the rearward portion of the flanking chambers 73, are single slots 105 so disposed to provide for a relatively modest flow of fluid in the lateral portions of the wider segment of the applicator pad 55.

The distribution plate 75′ may be formed such that the openings 100 extend from the upper surface and terminate at a distribution surface 76′. In such an embodiment, the applicator pad attachment surface 56 is strategically affixed to the distribution surface 76′ throughout its surface area by adhesive or other suitable affixation means known in the art, ensuring that the affixation means does not clog or otherwise occlude the openings 100. To further ensure that the openings will not be occluded by the adhesive or other affixation means, the distribution surface 76′ of the distribution plate 75′ may be recessed, as shown in FIGS. 18-19, so that the openings 100 terminate in the distribution surface 76′ of the distribution plate 75′ at a point spaced apart from and above the pad attachment surface 56. It is further contemplated that the outer perimeter of the bottom surface of the distribution plate 75′ may be formed with a downwardly projecting mounting ridge (not shown) for affixation of a corresponding in area portion of the perimeter of the applicator pad attachment surface 56 thereto.

With focus now on the internal construction of the housing 70′ in the alternate embodiment shown in FIGS. 16-17, it is also keeping with the invention that the rear dispenser housing wall 85 may be formed with a coupling assembly 145′ (FIG. 17) including a mounting socket 111 for complemental mating with the neck yoke 33 and neck 45 of the container 22. The mounting socket 111 is formed with an inlet device 148′, which in this embodiment includes a tubular inlet bore 112 that extends forwardly and downwardly through the rear wall 85 and maintains fluid communication with the flow chamber 71′. The inlet bore 112 is formed with at a bore abutment ridge 114 extending inwardly from the walls of the bore 112 and defining a transition between the distal extent of the inlet bore 112 and the proximal extent of the flow chamber 71′. In FIGS. 16-17, the flow control 132 is depicted as being located at this transition, however, it may be located at any point along fluid communication path 130 from the container 22 to the applicator pad 55. As shown in FIGS. 16 and 17, when the container 22 is received in the inlet bore 112, the distal extremity of the neck 45 will be abutted against this abutment ridge 114. In such an embodiment, the abutment ridge 114 is annular in shape, having a central opening defining a portion of the fluid communication path 130 for passing the fluid therethrough from the container 22 and its neck 45 to the flow chamber 71′.

As set forth in the above described embodiment, the bore 112 may be further formed in its proximal region with a plurality of lugs 162 spaced apart to define clearance slots 165 therebetween such that the studs 50 of the container neck 45 will be snapingly engaged behind respective lugs 162 in the bore 112 to secure the container 22 to the housing 70′ and its coupling assembly 145′. While a snap lock connection has been described, it is further contemplated that any appropriate connection means, such as a threaded engagement or a clamp type connection, may be employed to facilitate coupling of the container 22 to the dispenser housing 70′.

In operation, the user will secure the container 22 in the coupling assembly 145′ of the dispenser housing 70′ by aligning the yoke 33 in the mounting socket 111 and seating the container neck 45 in the inlet bore 112 to thereafter inwardly advance the neck 45 through the inlet bore 112 in an alignment such that the locking studs 50 will be secured behind respective lugs 162 as set forth above. This will also result in the alignment of the mating curvilinear surfaces of the cowling 86 and the container end wall 31. As shown in the preferred embodiment of FIGS. 16-17, by squeezing inwardly the walls of the container 22, a user will then cause the fluid therein to flow from the container reservoir 24, through the inlet bore 112 and neck 45, and to the flow chamber 71′, and more specifically, to the outwardly and forwardly angled rear portion of the introduction chamber 72. This initially directs the flow of fluid over the rear most array of slots 101 into contact with the longitudinally medial portion of the distribution plate 75′, and will further effect flow through the second set of slots 102 for dispensation therethrough. Fluid flow will then continue to the more forwardly positioned and laterally spaced slots 103 for a laterally spaced dispensing thereof, and further to the forward more closely spaced slots 104. The fluid flow, under continued pressure from the squeezed container 22, will then continue forwardly and spread laterally across the forwardly disposed respective outlet edges 93 and 94 of the corresponding ribs 82 and 83 to flow laterally, outwardly and rearwardly into the respective flanking chambers 73, to then be driven rearwardly under pressure to flow over the slots 105 to thus dispense a measured modest amount of fluid to the lateral most portions of the distribution plate 75′.

With reference to FIGS. 17-19, as the fluid is forced to the various slots 100-105 of the distribution plate 75′, it then continues through such slots in such distribution plate to the distribution surface 76′, which may be recessed and spaced apart from the applicator pad 55 to prevent occlusion of the slots. The fluid will then flow to the attachment surface 56 of the applicator pad 55, and then through the applicator pad 55 or through channels 59 formed therethrough to be dispersed on the applicator working surface 62. The user then may pass the head of the applicator across the surface to be treated thus applying the underside working surface 62 of the pad 55 thereto. When the readily available supply of fluid at the working surface 62 has depleted, the user may thereupon squeeze the container 22 or otherwise again repeat the above described sequence. After treatment of a desired surface is completed, or the fluid in the container 22 has been exhausted, the user will rotate neck 45 in inlet bore 112 to align the studs 50 with a corresponding clearance slot 165. He or she may then withdraw these studs 50 through the clearance slots 165 to effectuate release of the container 22 from the housing 70′, and replace the container 22 as set forth above.

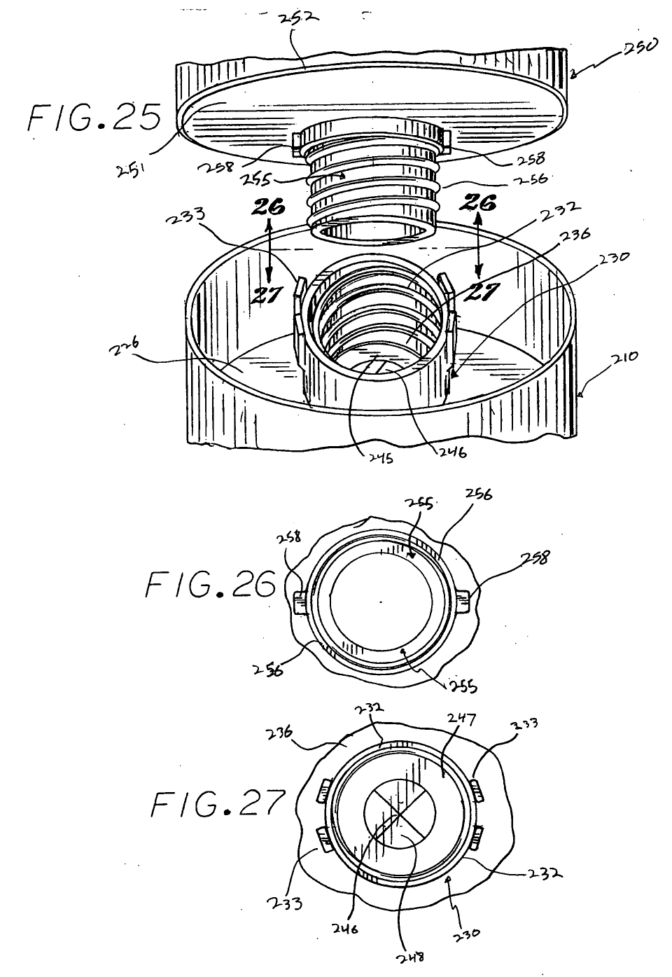

Referring to FIGS. 25-34, the fourth embodiment of the invention includes a dispenser housing 210 and container 250 having a construction similar to that shown in FIGS. 1-3, except for modification to the container coupling, chamber and chamber inlet. In this embodiment, the housing 210 includes a transverse wall 226 from which a tubular coupling assembly 220 projects for receiving the neck 255 of the container 250. An coupling includes an inlet tube 230, as shown in FIG. 25, projecting upwardly and outwardly from wall 226, and defines an open ended cavity 236 configured for complemental receipt of the neck 255 of the container 250 (FIG. 34).

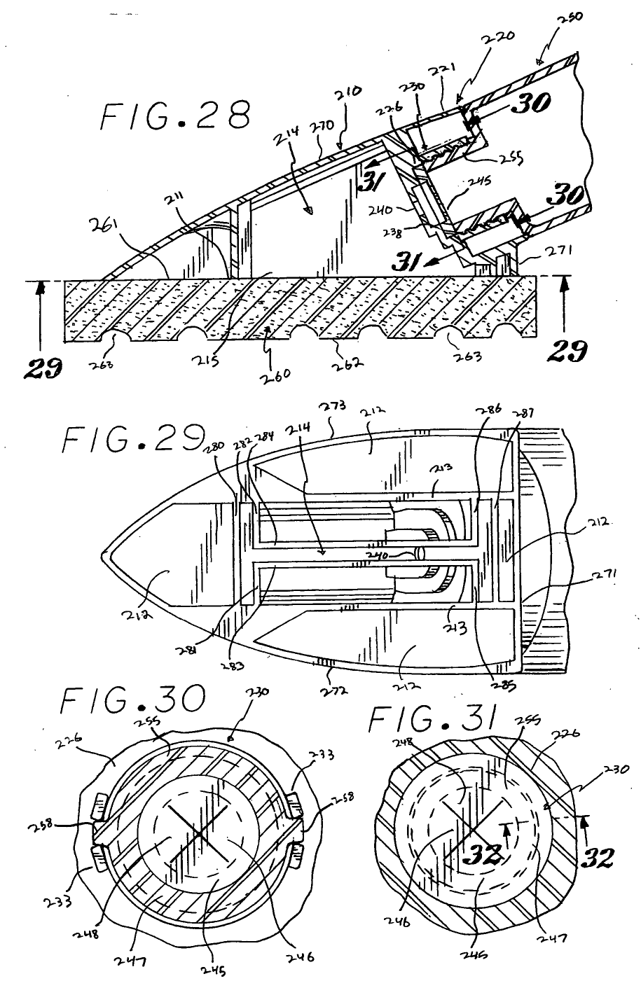

As shown in FIGS. 27 and 31-33, the coupling 220 is further formed in its bottom wall with a through inlet opening 240 surrounded by a gland 238 defining a rearwardly facing seat 239. Nested in the seat is a flow control device 245, which includes a flapper valve 246 to control the flow of fluid therethrough to the central opening 240 and then to a flow chamber 214. The valve 246 is circumscribed by a compressible seal 247, and may be slightly domed rearwardly. The valve is scored in a cruciform configuration to define flaps 248, which open inwardly in response to pressurized fluid flow to permit flow therethough. With this construction, the valve 246 also resists the return flow of fluid once it has passed through the flaps 248.

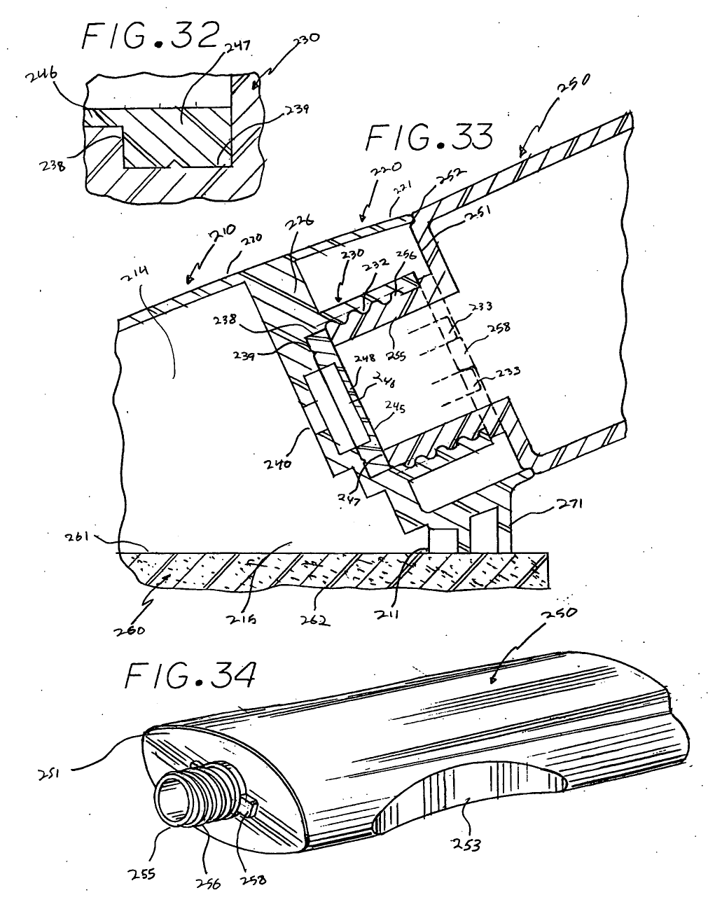

As described below, with the flow control 245 nested in the seat 239, as shown in FIG. 33, when the container neck 255 is advanced through the cavity 236 and the connector elements of the neck achieve a positive locking connection with the connector elements of the inlet, the open end of the neck 255 will be pressed against the flow control device 245 to form a fluid tight seal with the compressible seal 247.

The housing 210 is formed with a top wall 270, a rear wall 271 and side walls 272 and 273 that terminate in downwardly facing bottom edges disposed in a common plane to partially define the housing bottom surface 211, and may be internally formed with a central network of ribs and lightening holes. As shown in FIG. 29, the housing 210 further includes a plurality of vertically oriented longitudinal chamber ribs, shown for example at 280-287. One pair of these ribs (283 and 284) define the flow chamber 214 therebetween, with such chamber disposed in alignment with the opening 240 of the inlet 230. These ribs 280-287 terminate in downwardly facing bottom edges aligned in a common plane with the bottom edges of the housing walls to define the housing bottom surface 211, thus providing a mounting surface to which the attachment surface 261 of the applicator pad 260 is affixed or otherwise mounted. So mounted, the applicator pad cooperates with the chamber 214 to act as a distribution plate to distribute fluid along the attachment surface 261 to be metered downwardly through the pores of the pad to the working surface 262.

Thus, as depicted in FIGS. 29 and 33, after the fluid passes from the flow control 245 through inlet opening 240, it enters the flow chamber 214 to exit through a chamber outlet 215 to the attachment surface 236 of the applicator pad. In a preferred embodiment, the chamber outlet 215 defines an opening in the housing bottom surface 211 and as fluid passes through the outlet 215 it is thus simultaneously distributed in intimate contact along the attachment surface 261 of the applicator pad 260 for communication therethrough to the pad's working surface 262.

The chamber ribs, e.g. 280-288, cooperate to define any appropriately shaped and configured flow chamber 214 and chamber outlet 215 for communicating and distributing fluid along the longitudinal and lateral dimensions of the applicator pad as may be desired for a given application. In one exemplary embodiment, as depicted in FIG. 29, the chamber ribs combine to define a flow chamber 214 which is longitudinally elongated to form an “I” shaped chamber outlet 215.

The remaining portions of the housing bottom surface 211 that are not defined by the bottom edges of the housing walls 270-273 and chamber ribs 280-287 may be formed with any economical or functional configuration desired. For example, the remainder of the bottom surface 211 may be solid, or the housing may be further constructed with a plurality of longitudinally and/or laterally elongated reinforcing ribs 213 (FIG. 29) that terminate in bottom edges which form any appropriate bottom surface 211 pattern to provide surface area for bonding the attachment surface 261 of the pad 260 thereto.

As shown in FIG. 29, the housing bottom surface 211 may also be formed with a plurality of lightening cavities 212 conveniently situated about the periphery of the bottom surface and/or in the vicinity of the central portion adjacent to the chamber ribs 283 and 284. The cavities may be formed with varying and appropriate depths depending on the desired application and the financial and structural priorities established during the design and manufacturing processes. The contours of these cavities 212 may be defined by walls of varying convenient dimensions and orientations, or by the reinforcing ribs 213, and will also terminate in bottom edges to cooperate in defining the housing bottom surface 211 to which the pad 260 is mounted.

The pad 260 is conveniently constructed in the form of semi-open cell polymer sponge like material, which can be either formed by injection molding or cut from a stock of foam such as is well known in the art as being suitable for this purpose. Its dimensions and contours will be suited to a given application, but is preferably flat iron shaped and formed oversized relative to the plan view of the housing bottom surface 211 to project laterally outwardly therefrom to form respective peripheral skirts. In a preferred embodiment, the pad 260 is 3 inches wide at the base with the opposite edges curving forwardly and inwardly toward one another to a point located on the longitudinal center line to provide an overall length of 4¾ inches. The pad is preferably formed on its working surface 262 with undulations 263 (FIG. 28) in the form of sinusoidal grooves to facilitate uniform application of fluid.

To securely mount the container 250 in the housing 210 in a fluid tight sealing engagement, the container neck and inlet 230 are formed with connector elements which preferably facilitate a threaded engagement, as shown in FIGS. 25, 28 and 33.

In this embodiment, the container 250 (FIG. 34) is formed with a contoured groove formed about the periphery of the end wall 251 to define a forwardly facing contoured shoulder 252 configured to receive in a nesting relationship the complimentary shaped end of the coupling assembly 220 as shown in FIG. 33. The end wall 251 is preferably centrally formed with a tubular neck 255 to be received telescopically in the inlet coupling 230. The neck may take any convenient corresponding shape to that of the cavity for complemental receipt in the inlet 230, and in the preferred embodiment as shown in FIGS. 25 and 34, is tubular in shape for complemental receipt in the inlet tube 230. The neck 255 is further outwardly formed with screw threading 256, and is configured at its base with a pair of diametrically outwardly projecting neck lugs 258 situated on opposite sides of the neck. The exterior surface of the container 250 need not be specifically ergonomically adapted, however, as shown in FIG. 34, will preferably include finger grips 253, and may include other features such as a palm pad or the like.

For receiving the container 250 of this embodiment therein, as shown in FIGS. 25 and 33, the coupling assembly 20 of the housing 210 includes a cowling 221 formed for complemental mating with the container end wall 251 by nesting against the shoulder 252 when the neck 255 is fully received in the inlet 230. To this end, the inlet is formed with inwardly projecting threading 232 for establishing a threaded engagement with the neck threading 256, and with at least one resilient finger 233 extending upwardly beyond the outward termination of the inlet 230. In the embodiment of FIGS. 25 and 30, the inlet tube 230 is formed with the threading 232, and includes two pairs of resilient fingers 233 situated on diametrically opposite sides of the tube, with the fingers of each pair being spaced apart a distance sufficient thereof to receive a neck lug 258 between the free ends.

To enable mounting and locking of the container 250 into the inlet 230, as shown in the embodiment of FIGS. 25 and 33, the container neck 255 is telescopically received into the inlet tube 230 and, upon rotation of the container 250, the neck will rotate to engage the neck screw threads 256 with the inlet threads 232. Upon continued rotation and threaded mating, the neck 255 is advanced axially inwardly in the inlet tube 230 until the open end of the neck is abutted against the seal, and the neck lugs 258 engage respective free ends of the fingers 233 to flex such free ends of the engaged fingers 233 and, as rotation continues, cam therepast to register the lugs 258 between the respective finger pairs 233, as shown in FIG. 30.

With the lugs 258 registered between the fingers 233 of the finger pairs, the open end of the neck 255 will be seated against the compressible seal 247 and the fingers 233 will resist further clockwise or counterclockwise rotation of the container 250 to alert the operator sealing contact has been made. As shown in FIG. 33, this engagement will further facilitate the mating engagement of the cowling 221 with the container end wall 251 as the cowling is registered against the shoulder 252. To release the container from its engagement with the inlet, the user will simply rotate the container in the opposite direction to initially cause the lugs 258 to flex and clear the fingers 233 in the opposite direction from that described above, whereupon continued rotation will cause the neck threading 256 to disengage its threaded engagement with the inlet threading 232 and the container 250 may be removed from the housing 210.

While the inlet 230 has been described as tubular, as will be appreciated by those of skill in the art, the inlet and the mating neck 255 may take many different forms so long as they are configured for complemental mating, even to the extent of the chamber inlet being a neck and the container including a socket to receive the neck. Additionally, it is further contemplated that the neck 255 of container 250 may abut directly against the gland 238 and be seated in seat 239 when the neck is received in the inlet if the flow control device is located elsewhere along the fluid communication path leading from the container 250 to the applicator pad 260. Moreover, while the above described chamber ribs 280-287 have been described as defining an exemplary flow chamber 214, this is instructive of the manner in which the flow chamber can be constructed to direct fluid both longitudinally and laterally to the applicator pad's attachment surface, but is merely one contemplated configuration for accomplishing this objective. Additionally, while a single flow chamber 214 has been described in this embodiment, it is further contemplated that the housing 210 may include multiple flow chambers, or that individual chambers may be segmented into sub-chambers.

In this alternative embodiment, the container 350, as shown in FIG. 39, includes a contoured groove formed about the periphery of the end container wall 351 to define a forwardly facing contoured shoulder 352, and the end wall 351 is further is formed with a yoke 354 extending forwardly from the lower extend of the shoulder. The container neck 355 is centrally formed in the yoke to project outwardly from it, and includes a collar flange 356 that circumscribes the neck 355. The collar flange 356 extends rearwardly and outwardly to define a cam surface 357, and terminates by angling inwardly towards the neck 355 to define a locking surface 358. The container 350 need not be ergonomically configured, but preferably will include at least a pair of finger grips 353.

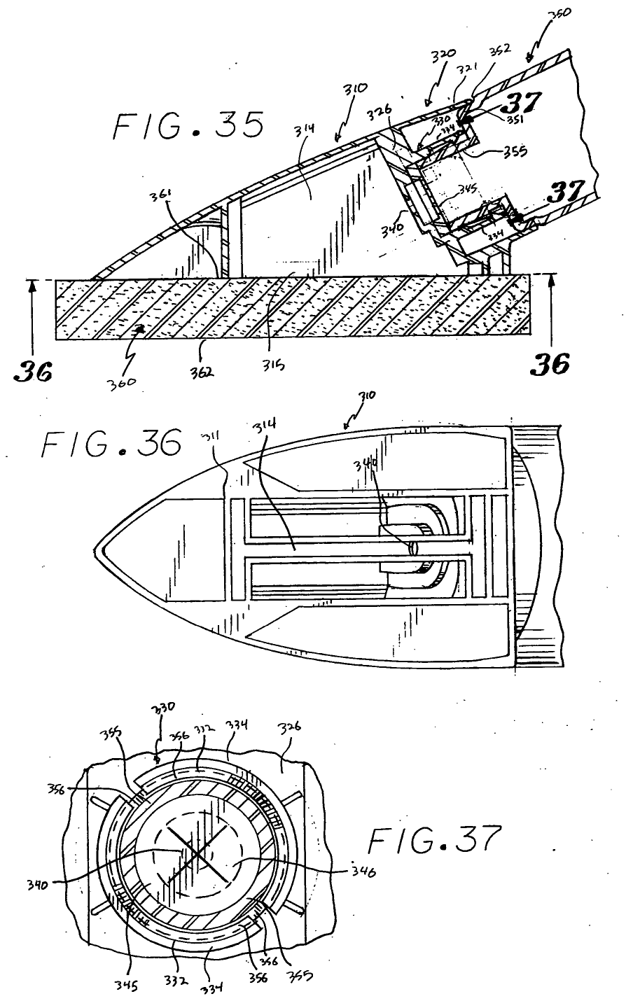

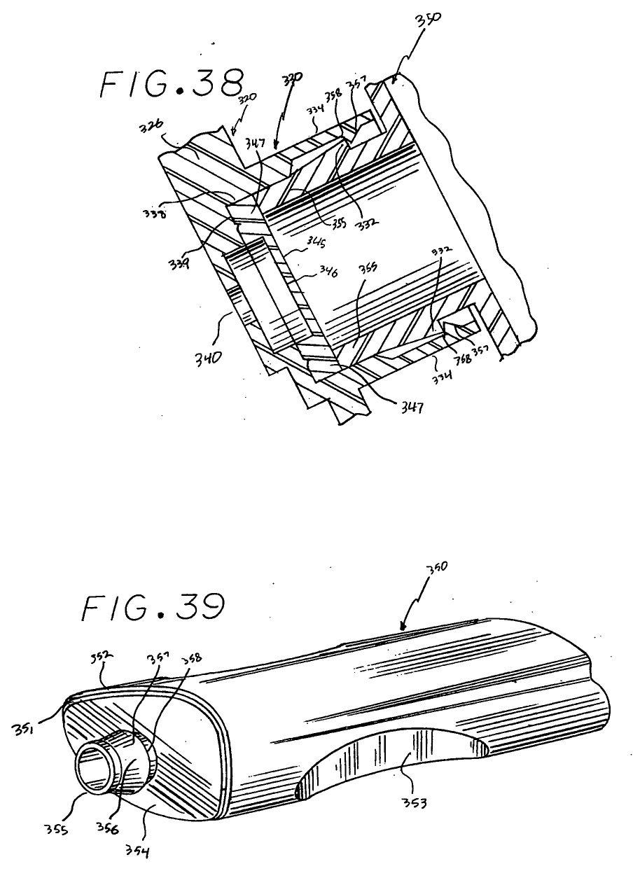

For receiving the container 350 of this embodiment therein, as shown in FIGS. 35 and 38, the housing 310 includes a coupling assembly 320 formed with a cowling 321 which terminates in its rear edge in a scallop configured on its top and bottom sides with rearwardly projecting curved tongues 322, which are configured to establish a nesting relationship with the shoulder 352 of the container end wall 351 when the yoke 354 is received in the coupling assembly 320.

Additionally, the housing 320 is formed with an inlet retaining assembly 326 from which an inlet device 330 projects rearwardly and upwardly. The retaining assembly 326 may take the form of a transverse wall, as shown in FIG. 35, or may be configured as a mounting block centrally disposed in the housing. The inlet 330 is defined by an inlet tube 331 which is formed with an inwardly projecting connector bead 332, and in the preferred configuration depicted in FIG. 37, the inlet tube 331 is segmented into two cantileverly projecting inlet tube half portions 334. separated by segmenting slots 335.

In this embodiment, each of the half portions 334 is formed in the vicinity of its distal extremity with its own inwardly projecting connector bead 332. The half portions 334 are rigid enough to reliably and securely hold the neck 355 of the container 350 when it is received therein. However, the segmenting slots 335 also lend sufficient resiliency to the half portions 334 to enable them to flex outwardly when receiving the container neck 355 and then to return to their original configuration for securing the neck in the inlet 148 after the connector beads 332 are registered behind the neck's collar flange 356. The inlet is further formed with a gland 338 having a rearwardly facing seat 339 and a centrally formed inlet opening 340, and a flow control device 345 including a valve 346 circumscribed by a compressible seal 347 is nested in the seat 339.

To enable mounting and locking of the container 350 into the inlet 330, as shown in FIGS. 37 and 38, the neck 350 is received in the cavity 336 between the half portions 334 and axially inwardly advanced in the inlet 330. Upon further advancement of the neck 350 through the cavity 336, the cam surface 357 of the neck collar flange 356 is slidingly engaged by the connector beads 332 of the shell portions 334 to cause the shell portions to flex outwardly. Further axial advancement of the neck 355 will then cause the beads 332 to clear the collar flange 356, at which time the shell portions 334 return to their original configuration to register the beads 332 behind the locking surface 358 of the flange 356 in the relationship shown in FIGS. 37 and 38.

So configured, the neck 355 will be received in the cavity 336 such that, with the beads 332 engaged securely behind the flange 356, the distal portion of the neck 355 will be sealingly engaged with the compressible seal 347 of the flow control device 354, as shown in FIG. 38, and will be securely seated in inlet 330. Thus, as shown in FIGS. 35 and 38, with the cowling tongues 322 nested against the forwardly facing shoulder 352 of the container end wall 351, the neck yoke 354 received in the coupling assembly 320, the neck 355 sealingly engaged with the compressible seal 347 and received in the inlet 330 and the beads 332 of the inlet tube half portions 334 registered securely behind flange 356 of the neck 355, the container 350 will be securely registered within the housing 310 in a close fit relationship to provide a fluid tight sealing engagement between the two.

To release the container 350 from the dispenser housing 310 and its coupling assembly 320, limited axial rotation of the container 350 and the cowling 321 relative to one another will disengage the complemental mating of the forwardly facing shoulder 352 of the container 350 and the rearward edges of the curved cowling tongues 322. This simultaneously rotates the neck 355 within the inlet 330. While rotating the neck 355, the disengaging force simultaneously exerted by the user to separate the container 350 from the housing 310 will meet resistance as the connector beads 332 slide against the flange 356. By continuing to exert such disengaging force, the half portions 334 will be flexed outwardly and the beads 332 will slide back over the flange 356 to release their locking engagement, thereby releasing the neck 355 from the inlet 330 and likewise the container 350 from the housing 310.

In the embodiment of FIGS. 35-39, the remaining construction does not differ significantly from that depicted in FIGS. 25-34. For example, FIG. 36 depicts a housing construction having a flow chamber 314 aligned with inlet opening 340 similar to the embodiment of FIG. 29, which includes a chamber outlet 315 opening onto the attachment surface 361 of pad 360 for communication of fluid therethough to working surface 362. The housing 310 is similarly formed with lightening cavities and chamber ribs and reinforcing ribs whose bottom edges terminate in a common plane to define the housing bottom surface 311 to which the pad 360 is attached.

In operation, for the shown FIGS. 25-29, the container will typically be sold filled with a vinyl treatment fluid and the neck sealed by a removable aluminum seal. The container may be unscrewed form the housing, the aluminum seal removed and the container 250 screwed back into the coupling assembly 220 dispenser housing 210. Rotation of the container and mating of the threads will advance the neck 255 in the coupling until the neck is abutted against the seal, at which time the neck lugs 258 are registered between the inlet's corresponding pairs of fingers 233, thereby resisting further rotation. This will further facilitate the mating engagement of the cowling 221 with the container end wall 251 to register the cowling against the shoulder 252.

By squeezing inwardly the walls of the container 250, a user will then cause the fluid therein to flow from the container, through the container neck 255 and inlet tube 230, through the valve 246 of the flow control 245, and then through the inlet opening 240 of the gland 238 to the flow chamber 214. As the pressurized fluid enters the flow chamber 214, some of the it will fall onto the portion of the attachment surface 261 of the applicator pad 260 situated below the inlet opening 240. The remainder may be distributed across the various inward surfaces of the chamber ribs 280-287 for communication to the remaining portions of the applicator pad underlying the chamber.

Thereafter, depending on the porosity of the chosen applicator pad 260, some portion of the deposited fluid will begin to flow through the pad. The remaining fluid will pool on the attachment surface 261 and will then be further distributed to other desired portions of the pad's longitudinal and lateral dimensions by flowing along the portion of the flow chamber 214 faced by the top surface of adjacent of the pad. In this manner, the fluid is distributed across the desired areas the attachment surface 261 for flowing downwardly through the pad 260 to the working surface 262 on the underside of the pad. As will be appreciated by those skilled in the art the chamber outlet may take many different forms, such as being elongated longitudinally as shown, elongated transversely or even a round opening, it only being important that fluid be communicated to the top of the pad so that pressurization of the container will force the fluid to the working surface to be distributed about the upholstery try to be treated.

After treatment of a desired surface is completed, or the fluid in the container 250 has been exhausted, the user may disconnect the container 250 from the housing 210 by rotating the container in the opposite direction until the neck lugs 258 are disengaged from their registration between the fingers 233. This will also cause the cowling 221 to disengage from its registration with the container shoulder 252. Continued rotation will withdraw the neck 255 out of the cavity 236 of the inlet tube 230 as the neck threading 256 disengages the inlet threading 232. This will release the container 250 from the housing 210, and the user may then replace the container 250 or refill it for subsequent applications.

While several particular forms of the invention have been illustrated and described, it will also be apparent to those skilled in the art that various modifications can be made without departing from the spirit and scope of the invention. Accordingly, it is not intended that the invention be limited except by the following claims.

Other features and advantages of the invention will become apparent from the following detailed description, taken in conjunction with the accompanying drawings which illustrate, by way of example, the features of the invention

1. An automotive appearance fluid applicator device for mounting to a fluid container of the type including a forwardly opening container outlet and a container coupling and comprising:
a housing including top and side walls and defining a flow chamber formed with a downwardly opening chamber outlet and configured in its rear portion with an inlet configured to mate with the container outlet and a housing coupling device for releasably coupling to the container coupling;
a flow control device for selectively restricting flow of the fluid from the container to the chamber;
an elongated porous applicator pad affixed on its top side to the housing and covering the outlet, the pad formed on its bottom side with a working surface and configured to receive the fluid from the outlet to provide for resistive flow therethrough to the working surface.

2. The applicator of claim 1 wherein:
the container coupling includes a fluid seal to cooperate with the container coupling to form a fluid tight seal.

3. The applicator of claim 1 for connection with a container formed with a neck defining the container outlet and wherein:
the housing is formed with a tube connected with the chamber configured to releasably receive the neck and including a seal cooperating with the neck to form a fluid tight seal.

4. The applicator of claim 3 for connection with a container having a neck formed with exterior screw threads and wherein:
the housing coupling device includes a tube including internal screw threads to cooperate with the exterior screw threads.

5. The applicator of claim 3 for connection with a container wherein the neck is formed around its periphery with a continuous collar having a peripheral surface which tapers rearwardly and inwardly to define a peripheral cam surface and wherein:
the tube includes an interior bead configured to, upon the neck being inserted in the tube, engage the peripheral cam surface and, upon further insertion, to ride outwardly and rearwardly relative thereto to engage therebehind.

6. The applicator as set forth in claim 5 wherein:
the tube is formed on its forward end with a reduced in cross-section shoulder defining a rearwardly facing seal and the bead is spaced from the forward end of the neck a distance sufficient to, when the bead is engaged behind the collar, urge the open end of the neck against the seal to cooperate in forming a fluid tight seal.

7. The applicator as set forth in claim 6 wherein
the flow control device includes a compression ring nested against the seal such that the open end of the neck is urged against the compression ring to form the fluid tight seal.

8. The applicator of claim 1 wherein:
the applicator pad is formed on its bottom surface with an undulating surface to facilitate uniform application of fluid.

9. An automotive appearance fluid applicator device including:
an applicator housing including top and side walls;
chamber means forming a chamber;
inlet means to receive fluid flow into the chamber;
outlet means in the bottom of the chamber flow fluid out of the chamber;
container means for containing fluid and container outlet means for flowing fluid out of the container means to the inlet means;
coupling means for releasably coupling the container to the applicator housing;
means for forcing fluid from the container means into the chamber; and
applicator pad means covering the chamber outlet for resistingly controlling the flow of fluid from the chamber outlet means and including a downwardly facing working surface to be rubbed over a surface to be treated with the fluid.

10. An automotive appearance fluid applicator device comprising:
a hand hold container formed with flexible walls and configured with a forwardly projecting neck formed on its exterior with a peripheral collar;
an applicator housing formed with top and side walls and configured with a fluid chamber having a downwardly opening outlet;
the housing further formed with an inlet tube connected on its forward end with the chamber and configured intermediately with a rearwardly facing shoulder mounting a compression seal and formed with a coupling shell configured on its interior with bead segments for engaging the peripheral collar, the shoulder being spaced from the bead a distance sufficient to cause the forward end of the neck, when the bead segments and collar are engaged, to sealingly engage the seal; and
a porous applicator pad covering the chamber outlet, formed with a downwardly facing working surface for metering fluid flow from the chamber to the working surface.

11. The applicator of claim 10 further including:
a flapper valve for controlling the flow of fluid to the chamber.

12. An automotive appearance fluid applicator device comprising:
a hand held container for containing a treatment fluid and including a forwardly opening container outlet;
an applicator housing formed by top and side walls and configured with a chamber having a downwardly opening chamber outlet, a rearward opening inlet communicating with the container outlet and a downwardly facing mounting surface;
a porous applicator pad mounted on the mounting surface and covering the chamber outlet to restrictively meter fluid flow from the chamber outlet to distribute flow to at least a portion of the area of the underside working surface of the pad; and
a coupling device including a first coupling element on the container and a second coupling element on the inlet for releasably coupling the container to the applicator housing.

13. The applicator device of claim 12 that includes:
a flow control device for restricting flow from the chamber to the container.

14. The applicator device of claim 12 wherein;
the first and second coupling elements are in the form of screw threads.

15. The applicator of claim 14 wherein:
the container is formed with a neck defining the container outlet; and
the housing is formed with a tube defining the inlet, the tube being reduced in diameter to form a rearwardly facing shoulder and the housing further including a compression seal on the shoulder configured to be engaged by the end of the neck to cooperate therewith in forming a fluid-tight seal.

16. The applicator of claim 15 wherein:
the coupling device includes a resilient catch device responsive to the neck being screwed a predetermined distance into the tube to engage and resist unscrewing of the container from the housing.

17. The applicator of claim 15 wherein:
the container includes at least one lug; and
the housing includes at least one finger for, upon the neck being screwed a predetermined distance into the tube, releasably engaging the lug to restrict rotation of the container relative to the tube.

18. The applicator of claim 15 wherein:
the container includes a pair of lugs; and
the housing includes two pair of resilient fingers projecting from the tube to, upon the neck being screwed a predetermined distance into the tube, engage respective ones of the lugs and, upon further screwing of the neck into the tube, to flex to clear the respective lugs so the respective lugs will be positioned between the respective pairs of fingers to restrict rotation of the container relative to the housing.

19. The applicator of claim 12 wherein:
the container is constructed to contain at least 8 fluid ounces of automotive appearance fluid.

20. The applicator of claim 12 wherein:
the application housing and pad are joined at a juncture disposed in a single plane.

21. The applicator of claim 12 wherein:
the pad is flat iron shaped having a base with a width of substantially 3 inches width the opposite sides tapering forwardly and inwardly to a point width located on a central axis having a length of substantially 4 3/9 inches.
